(19) United States
(12) Patent Application Publication
(10) Pub. No.: US 2022/0062132 A1
(43) Pub. Date: Mar. 3, 2022

(54) ODOR-STABLE ETHANOLIC PREPARATION CONTAINING OCTOCRYLENE

(71) Applicant: BEIERSDORF AG, Hamburg (DE)

(72) Inventors: Sarah SPROCK, Hamburg (DE); Annika KORTENHORN, Tornesch (DE)

(21) Appl. No.: 17/310,539

(22) PCT Filed: Jan. 29, 2020
(86) PCT No.: PCT/EP2020/052087

§ 371 Date: Aug. 10, 2021

(30) Foreign Application Priority Data
Feb. 13, 2019 (DE) 10 2019 201 861.0

(51) Int. Cl. A61K 8/40; A61K 8/34; A61K 8/36; A61Q 17/04

(52) U.S. Cl. CPC A61K 8/40; A61Q 17/04; A61K 8/36; A61K 8/34

(57) ABSTRACT

Cosmetic preparation containing: a) 2-ethylhexyl-2-cyano-3,3-diphenylacrylate (octocrylene), b) ethanol and c) propylheptyl caprylate (INCI).

The present invention relates to a cosmetic preparation containing 2-ethylhexyl 2-cyano-3,3-diphenylacrylate (octocrylene), ethanol and propylheptyl caprylate (INCI Propylheptyl Caprylate).

The trend away from pale complexion towards “healthy, athletic tanned skin” has continued for many years. To achieve this, people expose their skin to sunlight, since this causes pigment formation in the sense of melanin formation. However, the ultraviolet radiation of sunlight also has a harmful effect on skin. In addition to acute damage (sunburn), long-term damage such as an increased risk of developing skin cancer occurs on excessive irradiation with light from the UVB range (wavelength: 280-320 nm). Excessive exposure to UVB and UVA radiation (wavelength: 320-400 nm) additionally leads to a weakening of the elastic and collagen fibers in connective tissue. This leads to numerous phototoxic and photoallergic reactions and results in premature aging of the skin.

To protect the skin, a series of photoprotective filter substances have therefore been developed which may be used in cosmetic preparations. These UVA and UVB filters are in most industrialized countries collated in the form of positive lists such as Annex 7 of the Kosmetikverordnung [German Cosmetics Ordinance]

The plethora of commercially available sunscreens must not however obscure the fact that these prior art preparations have a number of disadvantages.

A UV filter that is widely used in daily care products and sunscreens is the compound 2-ethylhexyl 2-cyano-3,3-diphenylacrylate (octocrylene) which, in addition to its UV filter properties in the UVB range, is primarily used to stabilize the UVA filter 4-(tert-butyl)-4′-methoxy-dibenzoylmethane against photochemical degradation.

A disadvantage of the prior art is the fact that octocrylene in the presence of ethanol usually results in an “off-odor” in the preparations in the course of storage and also after a certain time when applied to skin. The resulting smell is vaguely reminiscent of Maggi seasoning familiar from cooking or the smell of lovage. The cause of this off-smell is the formation of sotolon (3-hydroxy-4,5-dimethyl-5H-furan-2-one), which has the following structure:

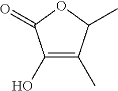

The reaction mechanism which leads to the formation of 3-hydroxy-4,5-dimethyl-5H-furan-2-one (hereinafter referred to as sotolon) is still unknown and not understood.

One possibility of avoiding the off-smell is, of course, to forego the use of ethanol. However, ethanol is increasingly being used to protect the “preservative-free” cosmetic preparations, so popular with consumers, from microbial contamination. In addition, ethanol is the ideal basis for transparent spray preparations, for example sunscreen sprays, due to its solvent properties, sensory properties and low price.

According to the prior art, attempts are therefore made in the case of ethanolic preparations to mask the olfactory off-note of the sotolon by using correspondingly high amounts of perfumes. However, this solution has the disadvantage that the perfumes evaporate from the preparation over time, whereas the concentration of sotolon increases over time. In addition, there is also a growing need in cosmetics for so-called “perfume-free” preparations in which odor-masking is then not possible.

It was therefore the object of the present invention to develop a cosmetic preparation (in particular a daily care product or a sunscreen) containing octocrylene and ethanol in which the formation of an off-odor and especially the formation of sotolon is suppressed.

Surprisingly, the object is achieved by a cosmetic preparation comprising

a) 2-ethylhexyl 2-cyano-3,3-diphenylacrylate (octocrylene)

b) ethanol and

c) propylheptyl caprylate (INCI: Propylheptyl Caprylate).

Although the prior art reveals DE102015214499.2, which discloses the use of lauryl alcohol diphosphonic acid for odor stabilization, and also DE 102007017440.5, DE 102007017434.0, DE 102007017536.3, DE 102007017438.3, DE 102007017435.9, DE 102007024348.2, DE 102017213232.9, which disclose the use of propylheptyl caprylate in cosmetics, these documents could still not indicate a route to the present invention.

It is advantageous according to the invention when the preparation contains 2-ethylhexyl 2-cyano-3,3-diphenyl acrylate (octocrylene) in a total amount of 0.1 to 10% by weight, based on the total weight of the preparation. In this case, a content from 3 to 9% by weight, based on the total weight of the preparation, is preferred in accordance with the invention.

It is also advantageous according to the invention when the preparation contains ethanol in a total amount of 0.1 to 65% by weight, based on the total weight of the preparation. In this case, an ethanol content of 3 to 60% by weight, based on the total weight of the preparation, is preferred in accordance with the invention.

Advantageous embodiments of the present invention are characterized in that the preparation contains propylheptyl caprylate at a concentration of 0.1 to 25% by weight, based on the total weight of the preparation. Here, the use concentration preferred according to the invention is from 0.5 to 10% by weight, based on the total weight of the preparation. Propylheptyl caprylate (IUPAC: 2-propylheptyl octanoate) can be purchased, for example, from BASF under the trade name Cetiol® Sensoft.

It is preferred according to the invention when the ratio by weight of octocrylene to propylheptyl caprylate is from 1:250 to 1:0.01.

The preparation according to the invention can advantageously be in two different embodiments: As a clear (transparent) ethanolic lipid solution and as an emulsion.

1) Clear ethanolic lipid solution

These embodiments of the present invention that are advantageous according to the invention are characterized in that the preparation is transparent.

According to the invention, a preparation is considered transparent if it is possible in daylight to look through a disposable cuvette (Brand, 2.5 ml, wavelength range: 220 nm-900 nm) filled with the preparation according to the invention with the naked eye. Characters (font type Arial, font size 10) that are located immediately behind the disposable cuvette should be recognizable and legible.

These embodiments of the present invention that are advantageous according to the invention are characterized in that the preparation contains less than 1% by weight water, based on the total weight of the preparation.

2) Emulsion

This advantageous embodiment according to the invention is preferably in the form of an 0/W emulsion.

Moreover, these emulsions are characterized as advantageous according to the invention in that the preparation comprises one or more emulsifiers selected from the group of compounds contains glyceryl stearate citrate, cetearyl alcohol, sodium cetearyl sulfate+glyceryl stearate, cetearyl sulfosuccinate, sodium stearoyl glutamate, polyglyceryl-3 methylglucose distearate, stearic acid, polyglyceryl-10 stearate, polyglyceryl-4 diisostearate/polyhydroxystearate/sebacate, potassium cetyl phosphate.

The concentration of emulsifier (total concentration) in this case is advantageously from 0.1% to 3% by weight, based on the total weight of the preparation.

Lastly, it is advantageous in the context of the present invention when the water content of the o/w emulsions is from 20% to 60% by weight, based on the total weight of the preparation.

It is advantageous according to the invention when the preparation comprises one or more UV filters selected from the group of compounds 2-phenylbenzimidazole-5-sulfonic acid and/or salts thereof; phenylene-1,4-bi s(2-benzimidazyl)-3,3′-5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-m ethyl-6-[2-methyl-3-[1,3,3,3-tetramethyl-1-[(trimethylsilyl)oxy]disiloxanyl]propyl]phenol; 3-benzylidenecamphor; ethylhexyl salicylate; terephthalidenedicamphorsulfonic acid and/or salts thereof; 2-ethylhexyl 4-(dimethylamino)benzoate; amyl 4-(dimethylamino)benzoate; di(2-ethylhexyl) 4-methoxybenzalmalonate; 2-hydroxy-4-methoxy-4′-methylbenzophenone; 2,2′-dihydroxy-4-methoxybenzophenone; 4-(tert-butyl)-4′-methoxydibenzoylmethane; homomenthyl salicylate (homosalate); 2-ethylhexyl 2-hydroxybenzoate; dimethicodiethylbenzalmalonate; 3-(4-(2,2-bis ethoxycarbonylvinyl)phenoxy)propenyl)methoxysiloxane/dimethylsiloxane-copolymer; 4-(tert-butyl)-4′-methoxydibenzoylmethane; hexyl 2-(4′-diethylamino-2′-hydroxybenzoyl)benzoate; dioctylbutylamidotriazone (INCI: Diethylhexyl Butamidotriazone); 2,4-bis[5-1(dimethylpropyl)benzoxazol-2-yl-(4-phenyl)imino]-6-(2-ethylhexyl)imino-1,3,5-triazine with (CAS No. 288254-16-0); 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

Embodiments of the present invention that are preferred according to the invention are characterized in that the preparation contains homomenthyl salicylate (homosalate) and/or ethylhexyl salicylate, the use of ethylhexyl salicylate being particularly preferred according to the invention.

If the preparation according to the invention contains homomenthyl salicylate (homosalate), it is advantageous according to the invention to use this substance at a concentration of 0.1% to 15% by weight, based on the total weight of the preparation.

If the preparation according to the invention contains ethylhexyl salicylate, it is advantageous according to the invention to use this substance at a concentration of 0.1% to 5% by weight, based on the total weight of the preparation.

If the preparation according to the invention contains 4-(tert-butyl)-4′-methoxy-dibenzoylmethane, it is advantageous according to the invention to use this substance at a concentration of 0.1% to 5% by weight, based on the total weight of the preparation.

It is advantageous in the context of the present invention when the preparation is free from propylparaben, butylparaben, methylisothiazolinone, chloromethylisothiazolinone, IPBC, DMDM hydantoin, dimethylol glycol, dimethylolurea, sodium hydroxmethylglycinate, BHT, 3-(4-methylbenzylidene)camphor and 2-hydroxy-4-methoxybenzophenone (INCI: Oxybenzone), isoamyl 4-methoxycinnamate, ethylhexyl 4-methoxycinnamate and cyclomethicone.

Embodiments of the present invention that are advantageous according to the invention are also characterized in that the preparation contains C12-15 alkyl benzoate, octyldodecanol, hydrogenated vegetable oil (INCI: Hydrogenated Vegetable Oil) and/or caprylic/capric acid triglycerides. In particular, the use of hydrogenated vegetable oil (INCI: Hydrogenated Vegetable Oil) is preferred according to the invention.

It is also advantageous according to the invention if the preparation contains one or more chelating agents from the group of the compounds
1-hydroxyethane-(1,1-diphosphonic acid)/HEDP
aminotrimethylenephosphonic acid/ATMP
diethylenetriaminepenta(methylenephosphonic acid)/DTPMP
ethylenediaminetetra(methylenephosphonic acid)/EDTMP
phosphonobutanetricarboxylic acid/PBTC
iminodisuccinate
sodium polyphosphate
tetrasodium pyrophosphate
hydroxamic acid
polygalacturonic acid
succinic acid
formic acid
malic acid
1-hydroxyethane-(1,1-diphosphonic acid)/HEDP
aminotrimethylenephosphonic acid/ATMP
diethylenetriaminepenta(methylenephosphonic acid)/DTPMP
ethylenediaminetetra(methylenephosphonic acid)/EDTMP
phosphonobutanetricarboxylic acid/PBTC
iminodisuccinate
sodium polyphosphate
tetrasodium pyrophosphate
hydroxamic acid
polygalacturonic acid
succinic acid
formic acid
malic acid
ethylendiaminetetraacetic acid (EDTA)
and/or alkali metal salts thereof and/or amine N-oxides thereof.

It is advantageous according to the invention if the preparation contains one or more polysaccharides selected from the group of gums. It is particularly preferred to use the compounds welan gum, sclerotium gum and/or cellulose gum or compounds from the group of alginates and carboxymethyl cellulose. According to the invention, particular preference is given to the use of cellulose gum and carboxymethyl cellulose.

In any case, it is advantageous according to the invention if the preparation contains polysaccharides in a total amount of 0.01% to 2% by weight, based on the total weight of the preparation.

There are also advantageous embodiments according to the invention which are characterized in that the preparation comprises polyvinyl alcohol and/or Triacontanyl PVP and/or acrylates/octylacrylamide copolymer.

Furthermore, it is advantageous according to the invention when the preparation contains one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone, 8-hexadecene-1,16-dicarboxylic acid, polidocanol, glycerylglucose, (2-hydroxyethyl)urea, vitamin E and derivatives thereof, hyaluronic acid and/or salts thereof, and/or licochalcone A.

In the context of the present invention, it is advantageous if the preparation contains one or more thickeners.

According to the invention, one or more thickeners are preferably selected from the group of the compounds ammonium acryloyldimethyltaurate/VP copolymer, ammonium acryloyldimethyltaurate/beheneth-25 methacrylate copolymer, hydroxypropyl cellulose vinylpyrrolidone/acrylic acid copolymer.

It is particularly advantageous to select the film-forming agents from the group of polymers based on polyvinylpyrrolidone (PVP).

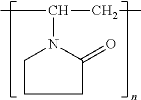

Particular preference is given to copolymers of polyvinylpyrrolidone, for example the PVP hexadecene copolymer and the PVP eicosene copolymer, which are obtainable under the trade names Antaron V216 and Antaron V220 from the GAF Chemicals Cooperation.

Further polymeric film-forming agents are also advantageous, such as sodium polystryrene sulfonate, which is obtainable under the trade name Flexan 130 from the National Starch and Chemical Corp., and/or polyisobutene, obtainable from Rewo under the trade name Rewopal PIB1000. Further suitable polymers are, for example, polyacrylamides (Seppigel 305), polyvinyl alcohols, PVP, PVP/VA copolymers, polyglycols, acrylate/octylacrylamide copolymer (Dermacryl 79). Likewise advantageous is the use of hydrogenated castor oil dimer dilinoleate (CAS 646054-62-8, INCI Hydrogenated Castor Oil Dimer Dilinoleate), which may be purchased from Kokyu Alcohol Kogyo under the name Risocast DA-H or also PPG-3 benzyl ether myristate (CAS 403517-45-3), which may be purchased under the trade name Crodamol STS from Croda Chemicals.

The use of acrylate/octylacrylamide copolymer (Dermacryl 79) is particularly preferred according to the invention.

Also according to the invention is a method for odor stabilization of ethanol-containing cosmetic preparations comprising 2-ethylhexyl 2-cyano-3,3-diphenylacrylate (octocrylene), characterized in that propylheptyl caprylate (INCI: Propylheptyl caprylate) is added to the preparation and the use of propylheptyl caprylate (INCI: Propylheptyl Caprylate) for the odor stabilization of ethanolic cosmetic preparations containing 2-ethylhexyl 2-cyano-3,3-diphenylacrylate (octocrylene).

The preparation according to the invention can advantageously be presented as a cream, lotion, gel, aerosol or spray.

### Comparative Experiment

The following formulations were prepared and their odor stability compared with one another:

| | | Sample 2 |
|---|---|---|
| | | (Sample 1 containing |
| | | propylheptyl caprylate |
| | Sample 1 | instead of butylene |
| | (Reference) | glycol dicaprylate/ |
| INCI | (m %) | dicaprate) m(%) |
| Dibutyl Adipate | 3 | 3 |
| C12-15 Alkyl Benzoate | 9.5 | 9.5 |
| Butylene Glycol Dicaprylate/ | 3 | 0 |
| Dicaprate | | |
| Propylheptyl Caprylate | 0 | 3 |
| Acrylates/Octylacrylamide | 1 | 1 |
| copolymer | | |
| Perfume | 0.7 | 0.7 |
| Glycerol | 2.5 | 2.5 |
| Alcohol Denat. | 50.3 | 50.3 |
| Homosalate | 9 | 9 |
| Octocrylene | 9 | 9 |
| Ethylhexyl Salicylate | 4.5 | 4.5 |
| Bis-Ethylhexyloxyphenol | 3 | 3 |
| Methoxyphenyl Triazine | | |
| Butyl Methoxydibenzoylmethane | 4.5 | 4.5 |

The samples were irradiated in glass vials under the following conditions:

Apparatus: Atlas Suntester

Filter combination: Coated quartz glass 56052388, and UV 56052371

Irradiation intensity: 765 W/m2

Exposure time: 72 h

The smell of the samples was then assessed by 5 experts: All 5 experts perceive a significantly more pronounced off-odor in sample 1 than in sample 2.

### EXAMPLES

The examples below are intended to illustrate the present invention without limiting it. Unless otherwise stated, all quantitative data, fractions, and percentages are based on the weight and the total amount or on the total weight of the preparations.

### Ex. 1: Transparent Sun Protection Spray SPF 20

| INCI | m(%) |
|---|---|
| Propylheptyl Caprylate | 2.00 |
| Isopropyl Palmitate | 3.00 |
| C12-15 Alkyl Benzoate | 10.00 |
| Acrylates/Octylacrylamide copolymer | 1.00 |
| Perfume | 0.70 |
| Glycerol | 4.00 |
| Alcohol Denat. | 56.30 |
| Homosalate | 9.00 |
| Octocrylene | 5.00 |
| Ethylhexyl Salicylate | 4.50 |
| Butyl Methoxydibenzoylmethane | 4.50 |

### Ex. 2: Transparent Sun Protection Spray SPF 50

| INCI | m(%) |
|---|---|
| Dibutyl Adipate | 3.00 |
| Propylheptyl Caprylate | 3.00 |
| C12-15 Alkyl Benzoate | 9.00 |
| Acrylates/Octylacrylamide copolymer | 1.80 |
| Perfume | 0.70 |
| Glycerol | 2.50 |
| Alcohol Denat. | 49.40 |
| Diethylamino Hydroxybenzoyl Hexyl Benzoate | 0.90 |
| Homosalate | 9.00 |
| Octocrylene | 9.00 |
| Ethylhexyl Salicylate | 4.50 |
| Bis-Ethylhexyloxyphenol Methoxyphenyl Triazine | 1.70 |
| Ethylhexyl Triazone | 1.00 |
| Butyl Methoxydibenzoylmethane | 4.50 |

### Example 3 Sun Protection Gel SPF50

| INCI | m(%) |
|---|---|
| Coco-glycerides | 0.50 |
| C12-15 Alkyl Benzoate | 5.50 |
| Acrylates/Octylacrylamide copolymer | 2.50 |
| Perfume | 0.80 |
| Glycerol | 3.00 |
| Propylheptyl Caprylate | 2.00 |
| Cetyl Alcohol | 5.00 |
| Hydroxypropyl Cellulose | 0.90 |
| Alcohol Denat. | 47.55 |
| Homosalate | 9.50 |
| Octocrylene | 9.00 |
| Ethylhexyl Salicylate | 4.50 |
| Bis-Ethylhexyloxyphenol Methoxyphenyl Triazine | 2.25 |
| Ethylhexyl Triazone | 2.25 |
| Butyl Methoxydibenzoylmethane | 4.75 |

### Ex. 4: Sun Protection Lotion SPF 30

| INCI | m(%) |
|---|---|
| Tocopheryl Acetate | 0.50 |
| Propylheptyl Caprylate | 1.00 |
| Hydroxyacetophenone | 0.40 |
| Panthenol | 1.40 |
| Ethylhexylglycerol | 0.50 |
| C12-15 Alkyl Benzoate | 4.50 |
| Myristyl Myristate | 1.00 |
| Hydrogenated Coco-Glycerides | 1.00 |
| Glyceryl Stearate Citrate | 2.00 |
| VP/Hexadecene Copolymer | 0.50 |
| Perfume | 0.40 |
| Glycerol | 7.00 |
| Sodium Hydroxide | 0.05 |
| Cetyl Alcohol | 0.30 |
| Stearyl Alcohol | 0.50 |
| Cellulose Gum | 0.10 |
| Acrylates/C10-30 Alkyl Acrylate Crosspolymer | 0.10 |
| Xanthan Gum | 0.30 |
| Aqua | 42.57 |
| Alcohol Denat. | 5.00 |
| Trisodium EDTA | 0.20 |
| Tetrasodium Iminodisuccinate | 0.10 |
| Homosalate | 9.00 |
| Octocrylene | 9.00 |
| Ethylhexyl Salicylate | 4.50 |
| Butyl Methoxydibenzoylmethane | 4.50 |
| Titanium Dioxide (nano) + Silica + Dimethicone | 3.00 |

1.-16. (canceled)

17. A cosmetic preparation, wherein the preparation comprises
(a) 2-ethylhexyl 2-cyano-3,3-diphenylacrylate (octocrylene)
(b) ethanol and
(c) propylheptyl caprylate.

18. The cosmetic preparation of claim 17, wherein the preparation comprises from 0.1% to 10% by weight of 2-ethylhexyl 2-cyano-3,3-diphenyl acrylate, based on a total weight of the preparation.

19. The cosmetic preparation of claim 17, wherein the preparation comprises from 0.1% to 65% by weight of ethanol, based on a total weight of the preparation.

20. The cosmetic preparation of claim 17, wherein the preparation comprises from 0.1% to 25% by weight of propylheptyl caprylate, based on a total weight of the preparation.

21. The cosmetic preparation of claim 17, wherein a ratio by weight of octocrylene to propylheptyl caprylate is from 1:250 to 1:0.01.

22. The cosmetic preparation of claim 17, wherein the preparation is transparent.

23. The cosmetic preparation of claim 17, wherein the preparation comprises less than 1% by weight water, based on a total weight of the preparation.

24. The cosmetic preparation of claim 17, wherein the preparation further comprises one or more UV filters selected from 2-phenylbenzimidazole-5-sulfonic acid and/or salts thereof; phenylene-1,4-bis(2-benzimidazyl)-3,3′-5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 3-benzylidene-camphor; ethylhexyl salicylate; terephthalidenedicamphorsulfonic acid and/or salts thereof; 2-ethylhexyl 4-(dimethylamino)benzoate; amyl 4-(dimethylamino)benzoate; di(2-ethylhexyl) 4-methoxybenzalmalonate; 2-hydroxy-4-methoxy-4′-methylbenzophenone; 2,2′-dihydroxy-4-methoxybenzophenone; 4-(tert-butyl)-4′-methoxydibenzoylmethane; homomenthyl salicylate (homosalate); 2-ethylhexyl 2-hydroxybenzoate; dimethicodiethylbenzalmalonate; 3-(4-(2,2-bis ethoxycarbonylvinyl)phenoxy)propenyl)methoxysiloxane/dimethylsiloxane-copolymer; 4-(tert-butyl)-4′-methoxydibenzoylmethane; hexyl 2-(4′-diethylamino-2′-hydroxybenzoyl)-benzoate; dioctylbutylamidotriazone (INCI: Diethylhexyl Butamidotriazone); 2,4-bis [5-1(dimethylpropyl)benzoxazol-2-yl-(4-phenyl)imino]-6-(2-ethylhexyl)imino-1,3,5-triazine (CAS No. 288254-16-0); 2,4-bis{ [4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris [anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

25. The cosmetic preparation of claim 17, wherein the preparation further comprises homomenthyl salicylate (homosalate).

26. The cosmetic preparation of claim 17, wherein the preparation further comprises ethylhexyl salicylate.

27. The cosmetic preparation of claim 17, wherein the preparation is free from propylparaben, butylparaben, methylisothiazolinone, chloromethylisothiazolinone, IPBC, DMDM hydantoin, dimethylol glycol, dimethylolurea, sodium hydroxmethylglycinate, BHT, 3-(4-methylbenzylidene)camphor and 2-hydroxy-4-methoxybenzophenone (INCI: Oxybenzone), isoamyl 4-methoxycinnamate, ethylhexyl 4-methoxycinnamate and cyclomethicone.

28. The cosmetic preparation of claim 17, wherein the preparation further comprises one or more of C12-15 alkyl benzoate, octyldodecanol, hydrogenated vegetable oil (INCI: Hydrogenated Vegetable Oil), caprylic/capric acid triglycerides.

29. The cosmetic preparation of claim 17, wherein the preparation further comprises one or more of
1-hydroxyethane-(1,1-diphosphonic acid)/HEDP
aminotrimethylenephosphonic acid/ATMP
diethylenetriaminepenta(methylenephosphonic acid)/DTPMP
ethylenediaminetetra(methylenephosphonic acid)/EDTMP
phosphonobutanetricarboxylic acid/PBTC
iminodisuccinate
sodium polyphosphate
tetrasodium pyrophosphate
hydroxamic acid
polygalacturonic acid
succinic acid
formic acid
malic acid
1-hydroxyethane-(1,1-diphosphonic acid)/HEDP
aminotrimethylenephosphonic acid/ATMP
diethylenetriaminepenta(methylenephosphonic acid)/DTPMP
ethylenediaminetetra(methylenephosphonic acid)/EDTMP
phosphonobutanetricarboxylic acid/PBTC
iminodisuccinate
sodium polyphosphate
tetrasodium pyrophosphate
hydroxamic acid
polygalacturonic acid
succinic acid
formic acid
malic acid
ethylendiaminetetraacetic acid (EDTA)
and/or alkali metal salts and/or amine N-oxides thereof.

30. The cosmetic preparation of claim 17, wherein the preparation further comprises one or more polysaccharides selected from gums.

31. The cosmetic preparation of claim 17, wherein the preparation further comprises one or more polysaccharides selected from welan gum, sclerotium gum, cellulose gum.

32. The cosmetic preparation of claim 17, wherein the preparation further comprises one or more alginates.

33. The cosmetic preparation of claim 17, wherein the preparation further comprises carboxymethyl cellulose.

34. The cosmetic preparation of claim 17, wherein the preparation further comprises polyvinyl alcohol and/or Triacontanyl PVP.

35. The cosmetic preparation of claim 17, wherein the preparation further comprises acrylate/octylacrylamide copolymer.

36. The cosmetic preparation of claim 17, wherein the preparation further comprises one or more compounds selected from alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone, 8-hexadecene-1,16-dicarboxylic acid, polidocanol, glycerylglucose, (2-hydroxyethyl)urea, vitamin E and derivatives thereof, hyaluronic acid and/or salts thereof, and/or licochalcone A.
